(19) United States
(12) Patent Application Publication
(10) Pub. No.: US 2026/0212600 A1
(43) Pub. Date: Jul. 23, 2026

(54) CASCADE REFINEMENT CORONARY ARTERY SEGMENTATION METHOD BASED ON GEOMETRIC DEFORMATION ENHANCEMENT

(71) Applicant: General Hospital of Northern Theater Command of the People's Liberation Army, Shenyang (CN)

(72) Inventors: Yuji Zhang, Shenyang, Liaoning (CN); Huishan Wang, Shenyang, Liaoning (CN); Guannan Liu, Shenyang, Liaoning (CN); Fangran Xin, Shenyang, Liaoning (CN); Huaiyang Liu, Shenyang, Liaoning (CN)

(21) Appl. No.: 19/312,200

(22) Filed: Aug. 27, 2025

(30) Foreign Application Priority Data
Jan. 17, 2025 (CN) 202510075620.X

(51) Int. Cl. G06T 17/20; G06T 5/70; G06T 7/00; G06T 7/10

(52) U.S. Cl. CPC G06T 17/20; G06T 5/70; G06T 7/0012; G06T 7/10; G06T 2207/10072; G06T 2207/20044; G06T 2207/20081; G06T 2207/20084; G06T 2207/20132; G06T 2207/30101

(57) ABSTRACT

A cascade refinement coronary artery segmentation method based on geometric deformation enhancement includes the following steps: S1, image acquisition; S2, image preprocessing; S3, mesh construction; S4, model construction; and S5, model training. In view of the problem that coronary arteries have complex anatomical structures, in the present disclosure, by integrating a geometric deformation network, a cascaded network is designed for coronary artery segmentation and vectorization of results, which can generate continuous and accurate coronary artery meshes, adapt to complex coronary artery structures, and avoid fragmentation of segmentation results. Different from mesh results generated by traditional voxel-based cube methods, the algorithm provided in the present disclosure can reconstruct finer vectorized coronary artery meshes with regular morphology, and avoid problems of bifurcation adhesion and point cloud dispersion in complex branches.

### CROSS-REFERENCE TO RELATED APPLICATION

This application claims priority of Chinese Patent Application No. 202510075620.X, filed on Jan. 17, 2025, the entire contents of which are incorporated herein by reference.

### TECHNICAL FIELD

The present disclosure relates to the cross technical field of deep learning and medical image segmentation, and in particular to a cascade refinement coronary artery segmentation method based on geometric deformation enhancement.

### BACKGROUND

Vascular disease is currently a common and serious health problem worldwide. It involves heart and blood vessel diseases caused by various factors, including hyperlipidemia, arteriosclerosis and hypertension, often leading to heart ischemia or hemorrhagic disease, which brings huge threat to human health. According to a special report on the Global Burden of Cardiovascular Disease released in 2023, annual deaths from cardiovascular disease has increased from 12.4 million to 19.8 million between 1990 and 2022, with deaths from coronary artery disease and its complications exceeding cancer. In China, cardiovascular disease morbidity and mortality are increasing year by year, bringing an increasing economic burden to residents and society, and has become a major public health problem. In this context, research on how to use deep learning algorithms to realize intelligent segmentation of coronary angiography images not only helps to improve the efficiency and accuracy of clinical diagnosis and treatment, but also helps to reduce the risk of misdiagnosis and missed diagnosis through standardized processes. It has great practical significance and broad application prospects for promoting cardiovascular disease early detection and timely intervention.

In recent years, with the development of deep learning technology, there has been a lot of research work to address the challenges of coronary artery segmentation. Park et al. provided a rank-based selective ensemble method, which aims to improve the performance of deep learning segmentation by combining weighted ensemble strategy with image-by-image quality estimation, and reduce morphological errors that affect fully automated quantitative analysis. Nasr-Esfahani et al. used convolutional neural network, vessel edge detection technique and threshold post-processing technique to realize the binary segmentation of coronary vessels. Samuel et al. provided a method, VSSCNet, for effectively segmenting blood vessels in coronary angiography using deep learning technology. The network achieves blood vessel localization on the preprocessed images through two vascular extraction layers with additional supervision and a unique feature map summation mechanism. Tmenova applied CycleGAN model to simulate and reconstruct angiographic images to increase the versatility of angiographic datasets. Ma provided a new method for dynamic coronary roadmapping using deep learning and Bayesian filters, which compensates for vessel motion caused by cardiac and respiratory activities in fluoroscopy through electrocardiograph (ECG) alignment and catheter tip tracking in X-rays; Mulay provided an edge adaptive instance normalization transfer technique based on deep learning for coronary artery segmentation, which combines adaptive instance normalization style transfer with dense extremum initiation network and convolutional block attention module to achieve the best coronary artery segmentation performance.

Coronary artery segmentation is the most critical step in coronary angiography image processing, segmentation accuracy directly determines the follow-up diagnostic effect. Even if there are many research works based on deep learning algorithms now, coronary artery segmentation still has the following technical difficulties:
1, complex anatomical structure: coronary arteries have a unique tree structure, which requires the segmentation algorithm to capture the entire vascular network from the trunk to small branches. The distribution of the tree-structured coronary arteries in three-dimensional space is non-uniform, and the diameter and direction of each branch are different, which increases the difficulty of segmentation. Meanwhile, the path of coronary arteries is tortuous, there are many sharp turns and stenotic regions, and the image features of these regions may be similar to those of surrounding tissues, making it difficult for the image-based segmentation method to distinguish blood vessels from surrounding tissues;
2, gray scale image with low contrast: in coronary angiographic images, blood vessels and surrounding background are expressed by gray values. Although the contrast agent injected into blood vessels can improve the visibility of blood vessels, the gray difference between blood vessel wall and image background is not significant. Especially in some complex situations, including coronary stenosis, calcification or hemodynamic changes, the blood vessel boundary may become blurred, which makes it difficult for segmentation algorithm to accurately identify blood vessel contours;
3, coronary artery vessel overlap: the coronary artery system of the heart has a complex three-dimensional structure, and different vessel branches overlap each other and intersect each other, which appears as an overlapping area in the two-dimensional projection angiographic image, increasing the processing difficulty of the segmentation algorithm. Since it is difficult to visually distinguish each vessel layer at the overlapping area, the segmentation technology needs to be able to effectively solve the problem of missing depth information and ensure accurate segmentation of each independent vessel segment; and
4, limitations of mesh deformation methods: existing mesh deformation methods mainly target large and regularly shaped organs, including liver and hippocampus. These methods are relatively effective in dealing with these organs because the geometric shapes of these organs are relatively simple, regular, and large in size. However, coronary arteries have a complex tree-like structure and many fine branches, which makes it difficult to directly apply the conventional mesh deformation methods to the segmentation of coronary arteries.

### SUMMARY

An objective of the present disclosure is to provide a cascade refinement coronary artery segmentation method based on geometric deformation enhancement. The method utilizes a deep topology reconstruction (DTR) algorithm to extract key points of coronary artery, regards a line connecting a starting point to an end point of each branch as a center line of a corresponding coronary artery branch, improves geometric accuracy and detail of segmentation by generating fine mesh annotations, and constructs a cascaded network in combination with UNet and graph convolution network (GCN). The UNet network is used for image segmentation to process vectorized segmentation of blood vessels.

In order to achieve the above objective, the present disclosure provides the following technical solutions. A cascade refinement coronary artery segmentation method based on geometric deformation enhancement includes the following steps:
S1, image acquisition: using coronary artery computed tomography angiography (CCTA) image data as an image data source, image data collection being from a public data set and a private data set, the public data set using an automated segmentation of coronary artery (ASOCA) data set, the private data set being a partial collected computed tomography angiography (CTA) image data set;
S2, image preprocessing: preprocessing an image before training, including four steps: image denoising, image cropping, image normalization and data enhancement;
S3, mesh construction: generating a whole framework of coronary artery fine mesh results, including three processes: skeletonization, reconstruction and integration, extracting key points of coronary artery tree and segmenting the key points into individual branches through the skeletonization, and reconstructing a smooth surface of each branch using these key points and coronary artery annotations;
S4: model construction: constructing a cascaded neural network based on geometry enhancement, including the steps of: training U-shaped network (UNet) and graph convolution network (GCN) jointly, guiding mesh deformation of the GCN using image features extracted by UNet, implementing mesh refinement segmentation from coarse to fine adopting a cascaded network strategy, the cascaded network strategy including two UNet-GCN connection networks, and executing mesh deformation and mesh refinement; and
S5: model training: using multiple loss functions for joint optimization, using Dice loss and Focal loss for joint optimization, and optimizing the GCN network with mesh losses, specifically including Chamfer distance loss, Laplacian smoothness loss, normal consistency loss, and edge loss.

Preferably, in S1, the ASOCA data set includes 40 training data and 20 test data in total, images of the data set have anisotropic resolution, with a planar resolution of 0.3-0.4 mm and a layer spacing of 0.625 mm, and the private data set includes 300 CTA image data, and images have isotropic resolution of 0.5 mm.

Preferably, in S2, the image preprocessing includes the specific steps of:
S2.1, image denoising: reducing random noise in the images, including detectors and electronic elements, and denoising the images using Gaussian filtering;
S2.1, image cropping: cropping the images, removing parts of the images that do not include coronary arteries, and uniformly cropping the images to a size [128, 128, 128];
S2.1, normalization processing: performing Z-score normalization on the cropped CTA images, and a Z-score normalization formula is as follows:

$$Z = ( x - μ ) / σ ;$$

and

where x is a hounsfield unit (HU) value of a pixel in a heart image, u is an average value of HU values of all pixels, and σ is a standard deviation of all pixels; and
S2.4, data enhancement: applying random contrast enhancement, random mirror flipping, random horizontal flipping, and random rotation to the data.

Preferably, in S3, the skeletonization, reconstruction and integration of the whole framework includes the specific steps of:
S3.1, skeletonization: extracting key points of the coronary artery and establishing a tree structure thereof using a deep reinforcement tree traversal agent algorithm, extracting a center line of the coronary artery, capturing a key of an anatomical structure of the coronary artery, and performing vessel enhancement on the image; constructing a linear structure similarity response function by analyzing properties of eigenvalues of a Hessian matrix, enhancing vessels in two-dimensional and three-dimensional images, followed by initializing the deep reinforcement tree traversal agent algorithm, and extracting the key points of the coronary artery and establishing the tree structure thereof through a traversal result of the deep enhancement model; and identifying the key points of the coronary artery tree by a deraining recursive transformer (DRT) algorithm, treating a straight line connecting a starting point and an end point of each branch as a center line of the corresponding coronary artery branch, and interpolating the center line of each branch using a cubic B-spline curve to smooth the result;
S3.2, reconstruction: performing reconstruction with a key point of each branch of the coronary artery, calculating a tangent line of the key point, and using the tangent line as a normal vector to form cross-sections of the coronary artery; on each cross-section, performing ray sampling at intervals of 15 degrees in a counterclockwise direction from the key point, intersecting with the coronary artery annotation to form a mesh layer of a boundary of the coronary artery, and applying a filter to smooth from the key point P_{Ki} to each boundary point

$${A}_{Ki}^{j} ,$$

where j represents an angle of the ray; and projecting a radius after smoothing onto the cross-section to form a boundary of the coronary artery on the cross-section, and after achieving the smooth boundary of the coronary artery on each cross-section, using boundary points of two adjacent cross-sections to create triangle patches to form a coronary artery mesh; and
S3.3, integration: merging the coronary artery meshes from each branch using a mesh Boolean operation to complete the entire coronary artery mesh, and assuming that a first input mesh is X and a second input mesh is Y, a union X∪Y of the two manifold meshes being a mesh that exists in X, in Y, or in both X and Y, and a combined result being a closed manifold mesh.

Preferably, in S3.1, the initializing the deep reinforcement tree traversal agent algorithm includes defining a state space, an action space and a reward function; and the state space includes local features of the blood vessel, the action space includes forward, backward and turning operations along the blood vessel path, and the reward function is designed according to connectivity and integrity of the blood vessel structure.

Preferably, in S3.2, the filter used is a one-dimensional Gaussian filter.

Preferably, in S4, the first network generates a coarse mesh result, which is used as an input of the second network, and the second network performs refinement deformation to generate a fine coronary artery mesh representation; and construction of a specific network model structure includes the steps of:
S4.1, in a first stage of the network, using a preprocessed cropped 3Dpatch block X∈ [symbol] ^{L×H̅×W}, training the UNet network to extract the image features of the coronary arteries, under the guidance of UNet projection image features, using the GCN to deform the mesh to achieve the vectorization of a segmentation result, and training the UNet and the GCN together;
S4.2, in a second stage of the network, fixing the network parameters of the previous UNet and directly using the network parameters to extract the image features of the coronary arteries, inputting the coarse mesh result of the coronary arteries into a new GCN without performing a pooling operation, cascading the two steps and generating a refined mesh result of the coronary arteries; and
S4.3. initializing a spherical mesh [symbol] ={ [symbol] , ε} as an input of the GCN network, where [symbol] represents a set of vertices and ε represents a set of edges; and replacing the network as a whole with a graph convolution residual block, which included by two graph convolution layers in a residual manner, and dimensions of input, hidden and output features are listed in parentheses of the GraphResBlock.

Preferably, in S4.3, the spherical mesh with 162 vertices and 480 edges is initialized for the sphere.

Preferably, in S5, the specific formula for co-optimization of Dice loss and Focal loss is as follows:

$${ℒ}_{UNet} = a {ℒ}_{D i c e} + b {ℒ}_{F o c a l}$$

where [symbol] _{Dice} is Dice loss, which is a loss function to measure an overlap between the prediction result and the real label, and is used for image segmentation tasks; [symbol] _{Focal} is Focal loss, which is used for dealing with the problem of category imbalance, and a and b represent weights of Dice loss and Focal loss;
further, the formula of Dice loss is specifically as follows:

$${ℒ}_{D i c e} = 1 - Dice , Dice = 2 \vert X ⋂ Y \vert / ( \vert X \vert + \vert Y \vert )$$

where X is a set of pixels in a segmentation result, and Y is a set of pixels in a real result; and the specific formula of Focal loss is as follows:

$${ℒ}_{F o c a l} = - ( α \cdot {( 1 - \hat{p} )}^{β} \cdot p \cdot log ( \hat{p} ) + ( 1 - α ) \cdot {\hat{p}}^{β} \cdot ( 1 - p ) \cdot log ( 1 - \hat{p} ) ) α$$

where α is a loss weight of foreground, a loss value contribution of the foreground during training may be adjusted by adjusting α, β is a adjustment factor, \hat{p} is a predicted value of the pixel in the target image sample, and p is a real value of the pixel in the target image sample.

Preferably, in S5, the overall mesh loss formula is as follows:

$${ℒ}_{G C N} = {λ}_{1} {ℒ}_{C D} + {λ}_{2} {ℒ}_{L a p} + {λ}_{3} {ℒ}_{N C} + {λ}_{4} {ℒ}_{E G}$$

where λ₁, λ₂, λ₃ and λ₄ are weights of loss terms, which are 0.7, 0.1, 0.1 and 0.1; [symbol] _{CD} is a Chamfer distance loss, which is used for measuring a distance between the predicted mesh and the real mesh and guiding the deformation of the mesh; the Chamfer distance includes two parts, one part is a sum of squares of distances from each point in the predicted mesh to the nearest point in the real mesh, and the other part is a sum of squares of distances from each point in the real mesh to the nearest point in the predicted mesh; and the specific formula is as follows:

$${ℒ}_{CD} ( {𝒱}_{1} , {𝒱}_{2} ) = \frac{1}{\vert {𝒱}_{1} \vert} {\sum }_{x ∈ {𝒱}_{1}} {min}_{y ∈ {𝒱}_{2}} {‖ x - y ‖}_{2}^{2} + \frac{1}{\vert {𝒱}_{2} \vert} {\sum }_{y ∈ {𝒱}_{2}} {min}_{y ∈ {𝒱}_{1}} {‖ x - y ‖}_{2}^{2}$$

[symbol] _{Lap} is used for smoothing the mesh, a uniform weight of all edges connected to one vertex is calculated to maintain the smoothness of the mesh, and the specific formula is as follows:

$${ℒ}_{L α p} = {\sum }_{𝒾 = 1}^{N} {\sum }_{j ∈ 𝒩 ( i )} {( {υ}_{i} - {υ}_{j} )}^{2}$$

where N is a number of vertices in the mesh, [symbol] (i) is a set of neighbor vertices of a vertex vᵢ, and vᵢ and vⱼ are positions of a vertex i and a vertex j;
[symbol] _{NC} is used for calculating an angle between each pair of adjacent face normals to maintain the smoothness and consistency of the mesh, and the specific formula is as follows:

$${ℒ}_{NC} = {\sum }_{ℯ ∈ ε} 1 - cos ( {𝓃}_{0} , {𝓃}_{1} )$$

where ε represents a set of edges, e is an edge, and n₀ and n₁ are adjacent normal vectors;
[symbol] _{EG} is used for calculating a length of each edge to avoid abnormal vertices, and the specific formula is as follows:

$${ℒ}_{E G} = {\sum }_{i = 1}^{M} {( {ℯ}_{i} - \overline{ℯ} )}^{2}$$

where M is a number of edges in the mesh, eᵢ is a length of the iₜₕ edge, and ē is an average length of all edges.

The present disclosure has the following advantageous effects over the prior art.
1. In view of the problem that coronary arteries have complex anatomical structures, in the present disclosure, by integrating a geometric deformation network, a cascaded network is designed for coronary artery segmentation and vectorization of results, which can generate continuous and accurate coronary artery meshes, adapt to complex coronary artery structures, and avoid fragmentation of segmentation results. Different from mesh results generated by traditional voxel-based cube methods, the algorithm provided in the present disclosure can reconstruct finer vectorized coronary artery meshes with regular morphology, and avoid problems of bifurcation adhesion and point cloud dispersion in complex branches.
2. In view of the problem of gray scale image and low contrast, in the present disclosure, the image features are extracted through the deep learning nnUNet network, and the initial spherical mesh is deformed by using the GCN to adapt to the complex geometric structure of the coronary artery. This method not only relies on the gray scale information of the image, but also utilizes the geometric information to enhance the accuracy of segmentation. The network can effectively extract and fuse features to improve the segmentation accuracy of coronary arteries, and maintain a high segmentation quality even when the vascular boundaries are unclear.
3. In view of the problem of coronary artery vessel overlap, in the present disclosure, multiple loss functions are used to optimize network training, the Chamfer distance loss is adopted to measure the distance between the predicted mesh and the real mesh, thereby guiding the deformation of the mesh. In addition, the present disclosure also uses the Laplacian smoothing loss and the normal consistency loss to ensure the smoothness and geometric consistency of the mesh, which is very effective for dealing with the overlapping areas of blood vessels. Through the joint optimization of these loss functions, the network of the present disclosure can learn more accurate geometric features, thereby distinguishing different vascular layers in the overlapping areas of blood vessels and providing clear and accurate segmentation results.
4. In view of the limitations of the mesh deformation method, in the present disclosure, firstly, the key points of the coronary artery are extracted through the depth-enhanced tree traversal agent, and the tree structure is established; each branch is reconstructed by using these key points and coronary artery annotation to form a smooth surface; when dealing with the complex multi-bifurcation of the coronary artery, each branch is integrated to form a more realistic vascular shape; and the network of the present disclosure can directly generate a detailed vectorized mesh from the voxel-based segmentation result without complex mesh deformation.

### BRIEF DESCRIPTION OF THE DRAWINGS

FIG. 1a is a schematic diagram of mesh layers forming coronary artery boundaries;

FIG. 1b is a schematic diagram of the coronary artery boundaries formed in cross-sections;

FIG. 1c is a schematic diagram of a mesh forming a coronary artery;

FIG. 1d is a rough schematic diagram of generated coronary artery meshes;

FIG. 1e is a schematic diagram of the coronary artery meshes smoothed along key points;

FIG. 2 is an architecture diagram of a cascaded neural network based on what enhancement;

FIG. 3 is a specific architecture diagram of a 3DUNetnetwork;

FIG. 4 is a schematic diagram of two-stage segmentation results of a cascaded network;

and

FIG. 5 shows results of coronary artery segmentation in different models.

### DETAILED DESCRIPTION

Technical solutions in the examples of the present disclosure will be described clearly and completely in the following with reference to the accompanying drawings in the examples of the present disclosure. Obviously, all the described examples are only some, rather than all examples of the present disclosure. Based on the examples in the present disclosure, all other examples obtained by those ordinary skilled in the art without creative efforts belong to the scope of protection of the present disclosure.

A cascade refinement coronary artery segmentation method based on geometric deformation enhancement includes the following steps.

In S1, image acquisition: in the present disclosure, the research content is coronary artery segmentation, and image data used is derived from CTA images; image data collection is from a public data set and a private data set, the public data set uses an ASOCA data set, the private data set is a partial collected CTA image data set; the ASOCA data set includes 40 training data and 20 test data in total, images of the data set have anisotropic resolution, with a planar resolution of 0.3-0.4 mm and a layer spacing of 0.625 mm; and the private data set includes 300 CTA image data, and images have isotropic resolution of 0.5 mm. These data sets are labeled with coronary arteries by professional radiologists, including two labels, no. 0 and No. 1 labels are background label and coronary artery label.

In S2, image preprocessing, before training the image, preprocessing is performed, including four steps: image denoising, image clipping, image normalization and data enhancement. The specific steps of image preprocessing are as follows.

In S2.1, image denoising: random noise is reduced in the images, including detectors and electronic elements, and the images are denoised using Gaussian filtering.

In S2.1, image cropping: the images are cropped, parts of the images that do not include coronary arteries are removed, and the images are uniformly cropped to a size [128, 128, 128].

In S2.1, normalization processing: Z-score normalization is performed on the cropped CTA images, and a Z-score normalization formula is as follows:

$$Z = ( x - μ ) / σ$$

where x is a hounsfield unit (HU) value of a pixel in a heart image, u is an average value of HU values of all pixels, and σ is a standard deviation of all pixels.

In S2.4, data enhancement: random contrast enhancement, random mirror flipping, random horizontal flipping, and random rotation are applied to the data.

In S3, mesh construction, in the present disclosure, generating a whole framework of coronary artery fine mesh results includes three processes: skeletonization, reconstruction and integration, key points of coronary artery tree are extracted and segmented into individual branches through the skeletonization, and a smooth surface of each branch is reconstructed using these key points and coronary artery annotations. When dealing with intricate coronary artery bifurcation, each branch is integrated to form a more realistic vessel shape.

In S3.1, skeletonization: in the present disclosure, key points of the coronary artery are extracted and a tree structure thereof is established using a DRT algorithm. Specifically, firstly, a center line of the coronary artery is extracted, a key of an anatomical structure of the coronary artery is captured, and vessel enhancement is performed on the image; and a linear structure similarity response function is constructed by analyzing properties of eigenvalues of a Hessian matrix, thereby enhancing vessels in two-dimensional and three-dimensional images, followed by the DRT algorithm is initialized, including defining a state space, an action space and a reward function; and the state space includes local features of the blood vessel, the action space includes forward, backward and turning operations along the blood vessel path, and the reward function is designed according to connectivity and integrity of the blood vessel structure. Finally, the key points of the coronary artery are extracted and the tree structure thereof is established through a traversal result of the deep enhancement model; and the key points of the coronary artery tree are identified by the DRT algorithm, a straight line connecting a starting point and an end point of each branch is treated as a center line of the corresponding coronary artery branch, and the center line of each branch is interpolated using a cubic B-spline curve to smooth the result.

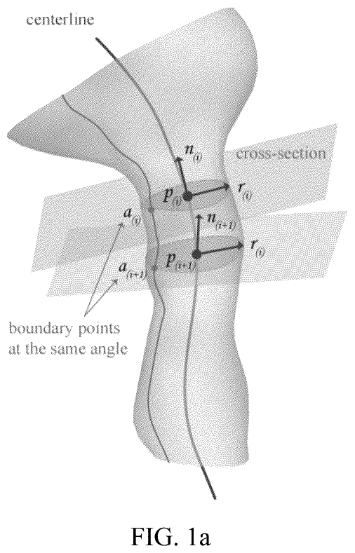

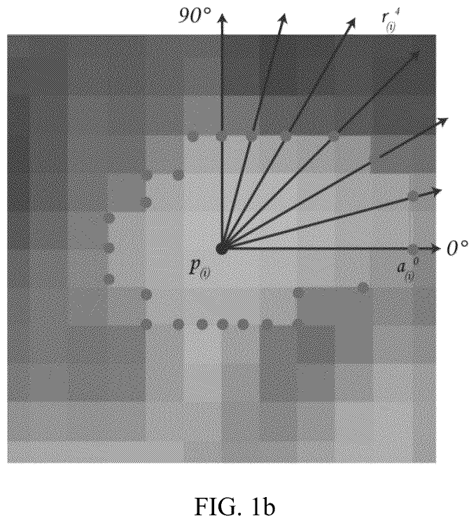

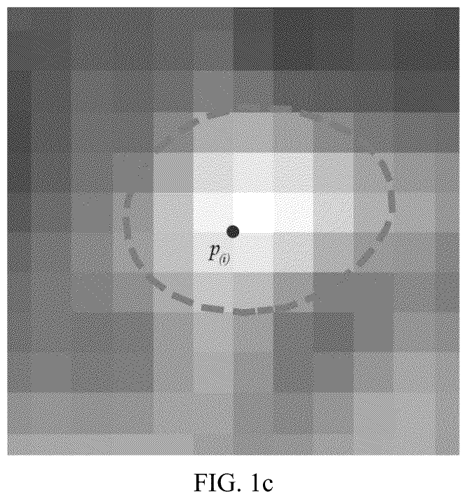

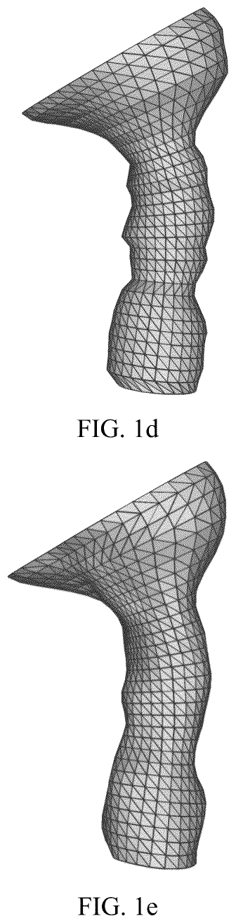

In S3.2, reconstruction: reconstruction is performed with a key point of each branch of the coronary artery, as shown in FIG. 1(a), a tangent line of the key point is calculated, and the tangent line is used as a normal vector to form cross-sections of the coronary artery; on each cross-section, ray sampling is performed at intervals of 15 degrees in a counterclockwise direction from the key point and intersected with the coronary artery annotation to form a mesh layer of a boundary of the coronary artery, as shown in FIG. 1(b), since voxel annotations are included by discrete voxels, the sparsity causes that coronary artery boundary sampling on the cross-section may appear rough and lack complete smoothness; and to restore the original morphology of the coronary artery as much as possible, a one-dimensional Gaussian filter is applied to smooth from the key point P_{Ki} to each boundary point

$${A}_{Ki}^{j} ,$$

where j represents an angle of the ray; and projecting a radius after smoothing onto the cross-section to form a boundary of the coronary artery on the cross-section, as shown in FIG. 1(c), after the smooth boundary of the coronary artery is achieved on each cross-section, triangle patches are created by using boundary points of two adjacent cross-sections to form a coronary artery mesh.

Although each boundary on the cross-section is flat, the generated coronary artery mesh is rough due to voxel-based discrete annotations, as shown in FIG. 1(c), the boundaries of adjacent coronary arteries vary greatly and lack seamless transition, which is far from the coronary arteries in reality, and in addition to the smoothing of the boundaries on the cross-section, the coronary artery mesh also needs to be flattened along the direction of the key points; according to the tubular structure of the coronary artery vessel, the line included by the boundary points sampled at the same angle is smoothed, e.g.

$$\{ … , {a}_{i}^{0} , {a}_{i + 1}^{0} , … \} ,$$

during smoothing, it is very important to determine the direction of the starting point, and in this way, the directions of the polar coordinate systems of adjacent cross-sections can be made as consistent as possible; and the starting ray from the previous cross-section is projected to the subsequent cross-section, the direction of the starting point is determined, the coronary artery mesh smoothed along the key points is shown in FIG. 1(e), and the generated coronary artery mesh not only preserves the geometric shape, but also is softer and closer to the real coronary artery.

In S3.3, integration: the coronary artery meshes of each branch are merged using a mesh Boolean operation to complete the entire coronary artery mesh, the mesh Boolean operation is performed on a closed manifold mesh, and assuming that a first input mesh is X and a second input mesh is Y, a union X∪Y of the two manifold meshes being a mesh that exists in X, in Y, or in both X and Y, and a combined result being a closed manifold mesh, which has a closed solid shape and includes the volume of all the input meshes, different from the previous method, the method of the present disclosure reconstructs and merges each branch mesh separately, instead of generating the entire coronary artery mesh as a whole; and this method helps to avoid complex modeling and greatly reduces the computational burden associated with coronary bifurcations, especially in the case of trifurcations, in addition since each branch of the bifurcation shares the same trunk, the transition between branches is smoother, and more closely approximates the actual structure of the coronary vessels.

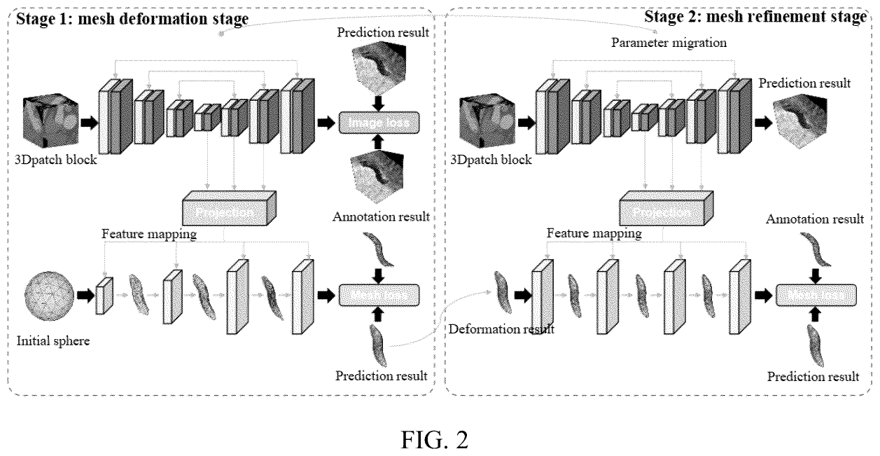

In S4: model construction: in order to be able to directly generate vectorized meshes of coronary arteries, the present disclosure provides a cascaded neural network based on geometric enhancement. The method includes jointly training UNet and graph convolutional networks, and using image features extracted by UNet to guide mesh deformation of GCN. In addition, the present disclosure adopts a cascaded network strategy to realize mesh refinement segmentation from coarse to fine, the cascaded network strategy is included by two UNet-GCN connection networks, which perform mesh deformation and mesh refinement. The first network generates a coarse mesh result, which is used as the input of the second network, the second network performs refinement deformation, and generate a fine coronary artery mesh representation; and the specific network model structure is shown in FIG. 2 below, and the specific steps are as follows.

In S4.1, in a first stage of the network, a preprocessed cropped 3Dpatch block X∈ [symbol] ^{L×H̅×W} is used, the UNet network is trained to extract the image features of the coronary arteries, under the guidance of UNet projection image features, the GCN is used to deform the mesh to achieve the vectorization of a segmentation result, and the UNet and the GCN are trained together.

In S4.2, in a second stage of the network, the network parameters of the previous UNet are fixed and directly used to extract the image features of the coronary arteries, the coarse mesh result of the coronary arteries is input into a new GCN without performing a pooling operation, the two steps are cascaded, and a refined mesh result of the coronary arteries is generated; and the specific parameters of the 3DUNet network structure are shown in Table 1 below, the ConvBlock included by two convolutional layers, max pooling is performed after the encoder-0, the encoder-1, the encoder-2 and the encoder-3, the step size is 2, in addition, the encoder-0 and the decoder-0, encoder-1 and Decoder-1, encoder-2 and Decoder-2 adopt jump connection, and the structure of 3DUNet is basically consistent with UNet.

TABLE 1
Detailed architecture diagram of 3DUNet network

| Layer | Output size | Network structure |
|---|---|---|
| Encoder-0 | 32 × 32 × 32 | Conv (3 × 3 × 3, 32) |
| Encoder-1 | 16 × 16 × 16 | ConvBlock (3 × 3 × 3, 64) |
| Encoder-2 | 8 × 8 × 8 | ConvBlock (3 × 3 × 3, 128) |
| Encoder-3 | 4 × 4 × 4 | ConvBlock (3 × 3 × 3, 256) |
| Decoder-3 | 4 × 4 × 4 | ConvBlock (3 × 3 × 3, 256) |
| Decoder-2 | 8 × 8 × 8 | ConvBlock (3 × 3 × 3, 128) |
| Decoder-1 | 16 × 16 × 16 | ConvBlock (3 × 3 × 3, 64) |
| Decoder-0 | 32 × 32 × 32 | ConvBlock (3 × 3 × 3, 32) |
| Classifier | 32 × 32 × 32 | Conv (1 × 1 × 1, 1) |

In S4.3, the specific structure of the graph convolution network is shown in Table 2 below. A spherical mesh [symbol] ={ [symbol] , ε} with 162 vertices and 480 edges is initialized as an input of the GCN network, where [symbol] represents a set of vertices and ε represents a set of edges. The network as a whole is replaced by a graph convolution residual block, which included by two graph convolution layers in a residual manner, and dimensions of input, hidden and output features are listed in parentheses of the GraphResBlock. After Graph-0 and Graph-1, a graph pooling operation is performed to divide each triangle into four triangles.

TABLE 2
Specific architecture diagram of GCN

Graph output

| Layer | Vertex | Surface | Network structure |
|---|---|---|---|
| Graph-0 | 162 | 320 | GraphResBlock (227, 192, 3) |
| Graph-1 | 642 | 1280 | GraphResBlock (419, 192, 3) |
| Graph-2 | 2562 | 5120 | GraphResBlock (419, 192, 3) |
| Graph-3 | 2562 | 5120 | GraphResBlock (192, 3) |
| Graph-4 | 2562 | 5120 | GraphResBlock (419, 192, 3) |
| FIG. -5 | 2562 | 5120 | GraphResBlock (419, 192, 3) |
| Graph-6 | 2562 | 5120 | GraphResBlock (419, 192, 3) |
| Graph-7 | 2562 | 5120 | GraphResBlock (192, 3) |

In S5: model training:

In order to train 3DUNet and GCN network together, the present disclosure uses multiple loss functions to co-optimize. Image loss is mainly voxel-based segmentation UNet, the Dice loss and the Focal loss are used to co-optimize, and the specific formula is as follows:

$${ℒ}_{UNet} = a {ℒ}_{D i c e} + b {ℒ}_{F o c a l}$$

where [symbol] _{Dice} is Dice loss, which is a loss function to measure an overlap between the prediction result and the real label, and is used for image segmentation tasks; [symbol] _{Focal} is Focal loss, which is used for dealing with the problem of category imbalance, and a and b represent weights of Dice loss and Focal loss;
further, the formula of Dice loss is specifically as follows:

$${ℒ}_{D i c e} = 1 - Dice , Dice = 2 \vert X ⋂ Y \vert / ( \vert X \vert + \vert Y \vert )$$

where X is a set of pixels in a segmentation result, and Y is a set of pixels in a real result; and the specific formula of Focal loss is as follows:

$${ℒ}_{F o c a l} = - ( α \cdot {( 1 - \hat{p} )}^{β} \cdot p \cdot log ( \hat{p} ) + ( 1 - α ) \cdot {\hat{p}}^{β} \cdot ( 1 - p ) \cdot log ( 1 - \hat{p} ) ) α$$

where α is a loss weight of foreground, a loss value contribution of the foreground during training may be adjusted by adjusting α, β is a adjustment factor, \hat{p} is a predicted value of the pixel in the target image sample, and p is a real value of the pixel in the target image sample.

The GCN network is optimized using mesh loss, which specifically includes Chamfer distance loss, Laplacian smoothing loss, normal consistency loss and edge loss. The overall mesh loss formula is as follows:

$${ℒ}_{G C N} = {λ}_{1} {ℒ}_{C D} + {λ}_{2} {ℒ}_{L a p} + {λ}_{3} {ℒ}_{N C} + {λ}_{4} {ℒ}_{E G}$$

where λ₁, λ₂, λ₃ and λ₄ are weights of loss terms, which are 0.7, 0.1, 0.1 and 0.1; [symbol] _{CD} is a Chamfer distance loss, which is used for measuring a distance between the predicted mesh and the real mesh and guiding the deformation of the mesh; the Chamfer distance includes two parts, one part is a sum of squares of distances from each point in the predicted mesh to the nearest point in the real mesh, and the other part is a sum of squares of distances from each point in the real mesh to the nearest point in the predicted mesh; and the specific formula is as follows:

$${ℒ}_{CD} ( {𝒱}_{1} , {𝒱}_{2} ) = \frac{1}{\vert {𝒱}_{1} \vert} {\sum }_{x ∈ {𝒱}_{1}} {min}_{y ∈ {𝒱}_{2}} {‖ x - y ‖}_{2}^{2} + \frac{1}{\vert {𝒱}_{2} \vert} {\sum }_{y ∈ {𝒱}_{2}} {min}_{y ∈ {𝒱}_{1}} {‖ x - y ‖}_{2}^{2}$$

[symbol] _{Lap} is used for smoothing the mesh, a uniform weight of all edges connected to one vertex is calculated to maintain the smoothness of the mesh, and the specific formula is as follows:

$${ℒ}_{L α p} = {\sum }_{𝒾 = 1}^{N} {\sum }_{j ∈ 𝒩 ( 𝒾 )} {( {υ}_{𝒾} - {υ}_{j} )}^{2}$$

where N is a number of vertices in the mesh, [symbol] (i) is a set of neighbor vertices of a vertex vᵢ, and vᵢ and vⱼ are positions of a vertex i and a vertex j;
[symbol] _{NC} is used for calculating an angle between each pair of adjacent face normals to maintain the smoothness and consistency of the mesh, and the specific formula is as follows:

$${ℒ}_{NC} = {\sum }_{e ∈ ε} 1 - cos ( {𝓃}_{0} , {𝓃}_{1} )$$

where ε represents a set of edges, e is an edge, and n₀ and n₁ are adjacent normal vectors;
[symbol] _{EG} is used for calculating a length of each edge to avoid abnormal vertices, and the specific formula is as follows:

$${ℒ}_{E G} = {\sum }_{𝒾 = 1}^{M} {( {ℯ}_{𝒾} - \overline{ℯ} )}^{2}$$

where M is a number of edges in the mesh, eᵢ is a length of the ith edge, and ē is an average length of all edges.

### Experimental Verification:

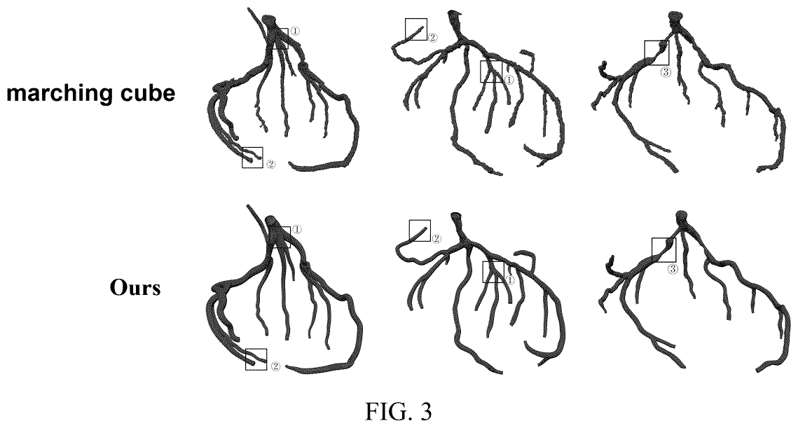

Based on the above method, the present disclosure carried out the following experiments. The first experiment is a vectorized mesh generation experiment, as shown in FIG. 3 below. The upper result is the reconstruction result obtained using the marching-cube method, and the lower result is the mesh generated by the algorithm of the present disclosure. The method of the present disclosure successfully reconstructs the smooth surface of the coronary artery, capturing rich details of small and narrow branches. In addition, the method of the present disclosure aims to handle various complex coronary artery structures, including three-bifurcation or even four-bifurcation, and the transition at the junction of multiple bifurcations appears natural and realistic. Further, the present method reconstruction accurately preserves the tubular morphology of the coronary arteries, even in cases where the vessels are compressed at the plaque, which ensures that the present method reconstruction closely resembles the true anatomy of the coronary arteries.

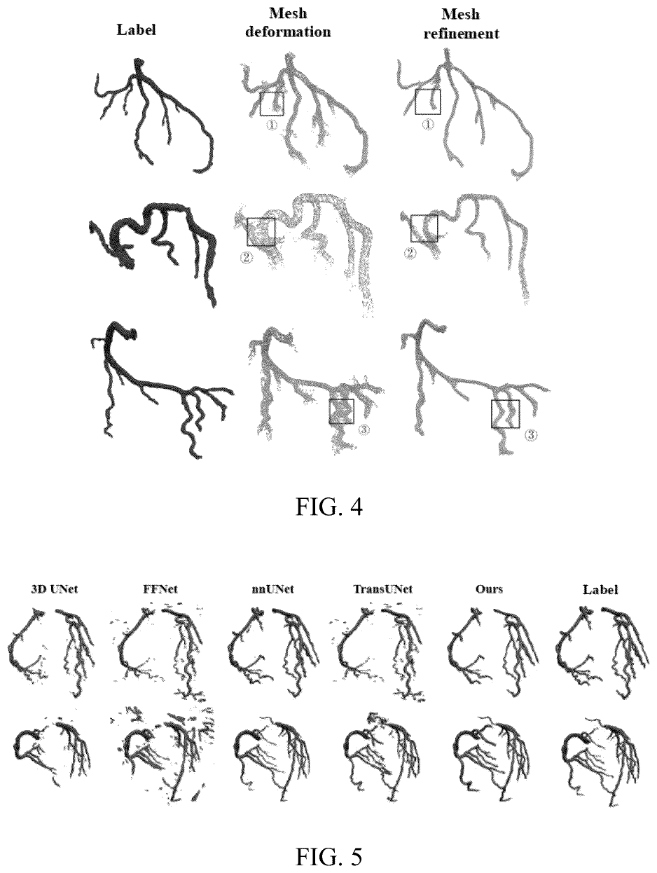

The second experiment is to test the output result of the cascaded network, as shown in FIG. 4 below, mesh deformation is the output result of the first stage, and mesh refinement is the output result of the second stage. It can be seen from the experimental results that the mesh deformation of the first stage cannot eliminate the interference between the blood vessels, causing the blood vessels to appear to stick together. Instead, the mesh refinement process effectively masks excess vessels, leaving only one central, larger vessel in the image input. This method ensures that adjacent parallel vessels do not stick to each other, thereby preventing mesh overlap.

The third experiment is to segment coronary arteries by different model methods. The experimental results are shown in FIG. 5 below. It can be seen from the experimental results that voxel-based segmentation often leads to vessel fragmentation, especially for coronary arteries with complex and twisted structures. In contrast, the geometry-based method of the present disclosure preserves the intact and complex coronary artery structure, effectively avoiding the problem of vessel rupture. Further, the method of the present disclosure accurately delineates tiny and narrow branches of coronary arteries, overcoming the limitations of sparseness and low resolution of CTA images, and the overall segmentation result of coronary arteries preserves the smoothness of vessels, resulting in more realistic morphology.

In the present disclosure, DTR algorithm is used to extract key points of coronary artery, based on these key points, the line connecting the starting point to the end point of each branch is considered as the center line of the corresponding coronary artery branch, and morphological regularization is provided for coronary artery segmentation; and by generating fine mesh annotations to improve the geometric accuracy and details of segmentation, a cascaded network is constructed to combine UNet and GCN, UNet network is used for image segmentation, and graph convolution network is used to deal with vectorization segmentation of blood vessels.

In the related art, extracting the centerline of the coronary artery is generally implemented by using the skeletonization algorithm. In this method, image segmentation or distance map generation needs to be performed first, which increases the calculation cost. Meanwhile, when processing such a complex anatomical structure as coronary artery, the entire vascular network from the trunk to the tiny branches cannot be captured. The present disclosure uses the DRT algorithm and utilizes the multi-task discriminator and the dynamic reward mechanism, which can effectively process the bifurcation points of the coronary artery, and can determine the tracking sequence without manual intervention. The multi-task discriminator is trained independently of the DRT agent, the distance from the point to the nearest bifurcation point and reference point can be calculated quickly, improving the accuracy of bifurcation point and end point detection.

In the related art, a complete coronary artery mesh is generated as a whole. This method will increase the computational burden of coronary artery bifurcations. Especially in the case of three bifurcations, the generated mesh cannot fit the coronary artery vessel situation. The present disclosure is completed in three steps of skeletonization, reconstruction, and merging. Reconstructing and merging each branch mesh effectively avoids complex modeling, and because each branch of the bifurcation shares the same trunk, the transition between branches is smoother and closer to the actual structure of coronary vessels, and rich details of more fine and narrow branches can be captured.

In the related art, most coronary artery segmentation uses a single UNet network or GCN network. This method cannot handle the situation where different vessel branches overlap and intersect each other, and cannot generate a coronary artery mesh with continuity and integrity, resulting in the problem of vessel fragmentation. In the present disclosure, the cascaded network is constructed, and the UNet and the graph convolution network are combined to achieve accurate segmentation and vectorization of coronary arteries. Through a segmentation strategy from coarse to fine, a coarse mesh result is first generated, and used as an input of a second network for refinement, which gradually improves the accuracy of segmentation. The UNet shares weights in the two stages, which ensures the consistency and accuracy of image feature extraction and accelerates the training speed of the network. By adding the use of GCN, the network can better utilize geometric features and improve the accuracy of segmentation and the smoothness of the mesh.

It is to be noted that in the present disclosure, relational terms including first and second, and the like, may be used herein to distinguish one entity or orientation from another entity or orientation without necessarily requiring or implying any actual such relationship or order between the entities or orientations. Furthermore, the terms “including”, “comprising”, or any other variations thereof, are intended to cover non-exclusive inclusion, and a process, method, article, or apparatus including a series of elements includes not only those elements, but also other elements not explicitly listed, or elements inherent to such process, method, article, or apparatus. With the implementation of the above scheme, the approximate realization of BTSH can be realized, and at the same time, the ability of ABTSH to ensure the minimum phase characteristics of discrete-time systems can also be guaranteed.

While examples of the present disclosure have been illustrated and described, those ordinary skilled in the art will understand that various changes, modifications, substitutions and variations can be made to these examples without departing from the principles and spirit of the present disclosure. The scope of the present disclosure is limited to the appended claims and equivalents thereof.

1. A cascade refinement coronary artery segmentation method based on geometric deformation enhancement, comprising the following steps:
S1, image acquisition: using coronary artery computed tomography angiography (CCTA) image data as an image data source, image data collection being from a public data set and a private data set, the public data set using an automated segmentation of coronary artery (ASOCA) data set, the private data set being a partial collected computed tomography angiography (CTA) image data set;
S2, image preprocessing: preprocessing an image before training, comprising four steps: image denoising, image cropping, image normalization and data enhancement;
S3, mesh construction: generating a whole framework of coronary artery fine mesh results, comprising three processes: skeletonization, reconstruction and integration, extracting key points of coronary artery tree and segmenting the key points into individual branches through the skeletonization, and reconstructing a smooth surface of each branch using these key points and coronary artery annotations;
S4: model construction: constructing a cascaded neural network based on geometry enhancement, comprising the steps of: training U-shaped network (UNet) and graph convolution network (GCN) jointly, guiding mesh deformation of the GCN using image features extracted by UNet, implementing mesh refinement segmentation from coarse to fine adopting a cascaded network strategy, the cascaded network strategy comprising two UNet-GCN connection networks, and executing mesh deformation and mesh refinement; and
S5: model training: using multiple loss functions for joint optimization, using Dice loss and Focal loss for joint optimization, and optimizing the GCN network with mesh losses, specifically comprising Chamfer distance loss, Laplacian smoothness loss, normal consistency loss, and edge loss.

2. The cascade refinement coronary artery segmentation method based on geometric deformation enhancement according to claim 1, wherein in S1, the ASOCA data set comprises 40 training data and 20 test data in total, images of the data set have anisotropic resolution, with a planar resolution of 0.3-0.4 mm and a layer spacing of 0.625 mm, and the private data set comprises 300 CTA image data, and images have isotropic resolution of 0.5 mm.

3. The cascade refinement coronary artery segmentation method based on geometric deformation enhancement according to claim 1, wherein in S2, the image preprocessing comprises the specific steps of:
S2.1, image denoising: reducing random noise in the images, comprising detectors and electronic elements, and denoising the images using Gaussian filtering;
S2.1, image cropping: cropping the images, removing parts of the images that do not comprise coronary arteries, and uniformly cropping the images to a size [128, 128, 128];
S2.1, normalization processing: performing Z-score normalization on the cropped CTA images, and a Z-score normalization formula is as follows:
$$Z = ( x - μ ) / σ$$
where x is a hounsfield unit (HU) value of a pixel in a heart image, u is an average value of HU values of all pixels, and σ is a standard deviation of all pixels; and
S2.4, data enhancement: applying random contrast enhancement, random mirror flipping, random horizontal flipping, and random rotation to the data.

4. The cascade refinement coronary artery segmentation method based on geometric deformation enhancement according to claim 1, wherein in S3, the skeletonization, reconstruction and integration of the whole framework comprise the specific steps of:
S3.1, skeletonization: extracting key points of the coronary artery and establishing a tree structure thereof using a deep reinforcement tree traversal agent algorithm, extracting a center line of the coronary artery, capturing a key of an anatomical structure of the coronary artery, and performing vessel enhancement on the image; constructing a linear structure similarity response function by analyzing properties of eigenvalues of a Hessian matrix, enhancing vessels in two-dimensional and three-dimensional images, followed by initializing the deep reinforcement tree traversal agent algorithm, and extracting the key points of the coronary artery and establishing the tree structure thereof through a traversal result of the deep enhancement model; and identifying the key points of the coronary artery tree by a deraining recursive transformer (DRT) algorithm, treating a straight line connecting a starting point and an end point of each branch as a center line of the corresponding coronary artery branch, and interpolating the center line of each branch using a cubic B-spline curve to smooth the result;
S3.2, reconstruction: performing reconstruction with a key point of each branch of the coronary artery, calculating a tangent line of the key point, and using the tangent line as a normal vector to form cross-sections of the coronary artery; on each cross-section, performing ray sampling at intervals of 15 degrees in a counterclockwise direction from the key point, intersecting with the coronary artery annotation to form a mesh layer of a boundary of the coronary artery, and applying a filter to smooth from the key point P_{Ki} to each boundary point
$${A}_{K i}^{j} ,$$
where j represents an angle of the ray; and projecting a radius after smoothing onto the cross-section to form a boundary of the coronary artery on the cross-section, and after achieving the smooth boundary of the coronary artery on each cross-section, using boundary points of two adjacent cross-sections to create triangle patches to form a coronary artery mesh; and
S3.3, integration: merging the coronary artery meshes from each branch using a mesh Boolean operation to complete the entire coronary artery mesh, and assuming that a first input mesh is X and a second input mesh is Y, a union X∪Y of the two manifold meshes being a mesh that exists in X, in Y, or in both X and Y, and a combined result being a closed manifold mesh.

5. The cascade refinement coronary artery segmentation method based on geometric deformation enhancement according to claim 4, wherein in S3.1, the initializing the deep reinforcement tree traversal agent algorithm comprises defining a state space, an action space and a reward function; and the state space comprises local features of the blood vessel, the action space comprises forward, backward and turning operations along the blood vessel path, and the reward function is designed according to connectivity and integrity of the blood vessel structure.

6. The cascade refinement coronary artery segmentation method based on geometric deformation enhancement according to claim 4, wherein in S3.2, the filter used is a one-dimensional Gaussian filter.

7. The cascade refinement coronary artery segmentation method based on geometric deformation enhancement according to claim 1, wherein in S4, the first network generates a coarse mesh result, which is used as an input of the second network, and the second network performs refinement deformation to generate a fine coronary artery mesh representation; and construction of a specific network model structure comprises the steps of:
S4.1, in a first stage of the network, using a preprocessed cropped 3Dpatch block X∈ [symbol] ^{L×H̅×W}, training the UNet network to extract the image features of the coronary arteries, under the guidance of UNet projection image features, using the GCN to deform the mesh to achieve the vectorization of a segmentation result, and training the UNet and the GCN together;
S4.2, in a second stage of the network, fixing the network parameters of the previous UNet and directly using the network parameters to extract the image features of the coronary arteries, inputting the coarse mesh result of the coronary arteries into a new GCN without performing a pooling operation, cascading the two steps and generating a refined mesh result of the coronary arteries; and
S4.3. initializing a spherical mesh [symbol] ={ [symbol] , ε} as an input of the GCN network, where [symbol] represents a set of vertices and ε represents a set of edges; and replacing the network as a whole with a graph convolution residual block, which comprised by two graph convolution layers in a residual manner, and dimensions of input, hidden and output features are listed in parentheses of the GraphResBlock.

8. The cascade refinement coronary artery segmentation method based on geometric deformation enhancement according to claim 7, wherein in S4.3, the spherical mesh with 162 vertices and 480 edges is initialized for the sphere.

9. The cascade refinement coronary artery segmentation method based on geometric deformation enhancement according to claim 1, wherein in S5, the specific formula for co-optimization of Dice loss and Focal loss is as follows:
$${ℒ}_{UNet} = a {ℒ}_{D i c e} + b {ℒ}_{F o c a l}$$
where [symbol] _{Dice} is Dice loss, which is a loss function to measure an overlap between the prediction result and the real label, and is used for image segmentation tasks; [symbol] _{Focal} is Focal loss, which is used for dealing with the problem of category imbalance, and a and b represent weights of Dice loss and Focal loss;
further, the formula of Dice loss is specifically as follows:
$${ℒ}_{D i c e} = 1 - Dice , Dice = 2 \vert X ⋂ Y \vert / ( \vert X \vert + \vert Y \vert )$$
where X is a set of pixels in a segmentation result, and Y is a set of pixels in a real result; and the specific formula of Focal loss is as follows:
$${ℒ}_{F o c a l} = - ( α \cdot {( 1 - \hat{p} )}^{β} \cdot p \cdot log ( \hat{p} ) + ( 1 - α ) \cdot {\hat{p}}^{β} \cdot ( 1 - p ) \cdot log ( 1 - \hat{p} ) ) α$$
where α is a loss weight of foreground, a loss value contribution of the foreground during training may be adjusted by adjusting α, β is an adjustment factor, \hat{p} is a predicted value of the pixel in the target image sample, and p is a real value of the pixel in the target image sample.

10. The cascade refinement coronary artery segmentation method based on geometric deformation enhancement according to claim 1, wherein in S5, the overall mesh loss formula is as follows:
$${ℒ}_{G C N} = {λ}_{1} {ℒ}_{C D} + {λ}_{2} {ℒ}_{L a p} + {λ}_{3} {ℒ}_{N C} + {λ}_{4} {ℒ}_{E G}$$
where λ₁, λ₂, λ₃ and λ₄ are weights of loss terms, which are 0.7, 0.1, 0.1 and 0.1; [symbol] _{CD} is a Chamfer distance loss, which is used for measuring a distance between the predicted mesh and the real mesh and guiding the deformation of the mesh; the Chamfer distance comprises two parts, one part is a sum of squares of distances from each point in the predicted mesh to the nearest point in the real mesh, and the other part is a sum of squares of distances from each point in the real mesh to the nearest point in the predicted mesh; and the specific formula is as follows:
$${ℒ}_{CD} ( {𝒱}_{1} , {𝒱}_{2} ) = \frac{1}{\vert {𝒱}_{1} \vert} {\sum }_{x ∈ {𝒱}_{1}} {min}_{y ∈ {𝒱}_{2}} {‖ x - y ‖}_{2}^{2} + \frac{1}{\vert {𝒱}_{2} \vert} {\sum }_{y ∈ {𝒱}_{2}} {min}_{y ∈ {𝒱}_{1}} {‖ x - y ‖}_{2}^{2}$$ [symbol]
_{Lap} is used for smoothing the mesh, a uniform weight of all edges connected to one vertex is calculated to maintain the smoothness of the mesh, and the specific formula is as follows:
$${ℒ}_{L α p} = {\sum }_{𝒾 = 1}^{N} {\sum }_{j ∈ 𝒩 ( 𝒾 )} {( {υ}_{𝒾} - {υ}_{j} )}^{2}$$
where N is a number of vertices in the mesh, [symbol] (i) is a set of neighbor vertices of a vertex vᵢ, and vᵢ and vⱼ are positions of a vertex i and a vertex j; [symbol]
_{NC} is used for calculating an angle between each pair of adjacent face normals to maintain the smoothness and consistency of the mesh, and the specific formula is as follows:
$${ℒ}_{NC} = {\sum }_{e ∈ ε} 1 - cos ( {𝓃}_{0} , {𝓃}_{1} )$$
where ε represents a set of edges, e is an edge, and n₀ and n₁ are adjacent normal vectors; [symbol]
_{EG} is used for calculating a length of each edge to avoid abnormal vertices, and the specific formula is as follows:
$${ℒ}_{E G} = {\sum }_{i = 1}^{M} {( {ℯ}_{i} - \overline{ℯ} )}^{2}$$
where M is a number of edges in the mesh, eᵢ is a length of the iₜₕ edge, and ē is an average length of all edges.
